(19) United States
(12) Patent Application Publication
(10) Pub. No.: US 2007/0129553 A1
(43) Pub. Date: Jun. 7, 2007

(54) Process for the preparation of glyceraldehyde acetonide

(76) Inventors: Peter Jan Leonard Mario Quaedflieg, Waalre (NL); Danniel Adrianus Franciscus Jacobus Boxtel Van, Weert (NL); Paulus Lambertus Alsters, Maastricht (NL); Peter Pojarliev, Wien (AT); Walther Gunther Jary, Steinbach am Attersee (AT)

Correspondence Address: NIXON & VANDERHYE, PC, 901 NORTH GLEBE ROAD, 11TH FLOOR, ARLINGTON, VA, 22203, US

(21) Appl. No.: 10/576,447

(22) PCT Filed: Oct. 25, 2004
(86) PCT No.: PCT/EP04/12064

§ 371 Date: Jul. 14, 2006

(30) Foreign Application Priority Data
Oct. 28, 2003 (EP) 03078392.2

(51) Int. Cl. C07D 317/08; C07D 211/54

(52) U.S. Cl. 549/229; 546/216

(57) ABSTRACT

The invention relates to a process for the preparation of glyceraldehyde acetonide by oxidation of 2,2-dimethyl-1,3-dioxolane-4-methanol by an oxidizing agent, wherein the 2,2-dimethyl-1,3-dioxolane-4-methanol is oxidized by an organic N-chloro compound in the presence of an inert base and TEMPO or a TEMPO-derivative. In one embodiment of the invention enantiomerically enriched glyceraldehyde acetonide is prepared from the corresponding enantiomerically enriched 2,2-dimethyl-1,3-dioxolane-4-methanol. Preferably, the organic N-chloro compount is trichloroisocyanuric acid or dichlorodimethyl hydantoin. Preferably, the inert base is sodium acetate or sodium bicarbonate.

The invention relates to a process for the preparation of glyceraldehyde acetonide by oxidation of the corresponding 2,2-dimethyl-1,3-dioxolane-4-methanol by an oxidizing agent.

Such a process is known from Ermolenko et al. 2001, Synlett, 1565-1566 “An expedient one-step preparation of (S)-2,3-O-isopropylidene-glyceraldehyde”. Ermolenko et al. disclose that (S)-glyceraldehyde acetonide can be prepared by oxidizing (R)-2,2-dimethyl-1,3-dioxolane-4-methanol with pyridinium chlorochromate (PCC) or pyridinium dichromate (PDC) in dichloromethane. A major disadvantage of this process is that very low yields (of 30%) are obtained due to severe by-product formation.

It is therefore the object of the invention to provide a process for the preparation of glyceraldehyde acetonide by oxidation of 2,2-dimethyl-1,3-dioxolane-4-methanol, wherein higher yields of glyceraldehyde acetonide are obtained.

This object is surprisingly achieved by oxidizing 2,2-dimethyl-1,3-dioxolane-4-methanol by an organic N-chloro compound in the presence of an inert base and TEMPO or a TEMPO-derivative of formula 1, 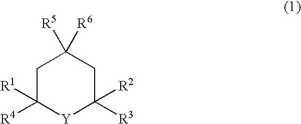
wherein R¹, R², R³ and R⁴ each independently stand for an alkyl group with 1 to 6 C-atoms and wherein R⁵ and R⁶ either both stand for H or an alkoxy group with 1 to 6 C-atoms or one stands for H and the other stands for an alkoxy group with 1 to 6 C-atoms, an alkylcarbonyloxy group with 1 to 6 C-atoms, an arylcarbonyloxy group with the carbonyloxy group having 1 to 6 C-atoms or an alkylcarbonylamino group with 1 to 6 C-atoms; or wherein R⁵ and R⁵ together stand for ketal groups of formula a-c 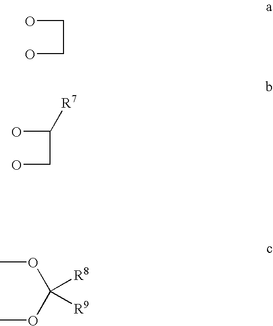
wherein R⁷ stands for an alkyl group with 1 to 6 C-atoms and R⁸ and R⁹ each independently stand for H or an alkyl group with 1 to 6 C-atoms and wherein Y stands for a group of general formula d-f 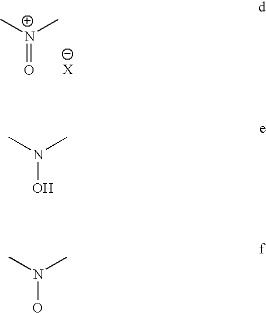
wherein X⁻ stands for an anion.

With this process a higher yield is achieved and substantially less by-products are formed. Additionally, the process of the invention does not require the environmentally unfriendly oxidizing agents PCC or PDC.

The fact that primary and secondary alcohols can be oxidized to the corresponding aldehydes by an organic N-chloro compound in the presence of TEMPO or a TEMPO-derivative and a base is known from EP-B-0 775 684 and is shown for a specific set of alcohols. However, it is surprising that specifically this oxidation method works so well in the oxidation of 2,2-dimethyl-1,3-dioxolane-4-methanol to glyceraldehyde acetonide. This is surprising, because many other methods to oxidize alcohols are known and none of them has been found to be suitable for the oxidation of 2,2-dimethyl-1,3-dioxolane-4-methanol to glyceraldehyde acetonide in reasonable yields (see Table A).

Preferably, the process of the invention is performed in the presence of TEMPO (2,2,6,6-tetramethylpiperidinyloxy radical).

In one embodiment of the invention, enantiomerically enriched glyceraldehyde acetonide is prepared from the corresponding enantiomerically enriched 2,2-dimethyl-1,3-dioxolane-4-methanol. The enantiomerically enriched glyceraldehyde acetonide can either be (S)-glyceraldehyde acetonide or (R)-glyceraldehyde acetonide. Preferably, the glyceraldehyde acetonide has an enantiomeric excess (ee)>80%, more preferably >90%, in particular >95%, more in particular >98%, most in particular >99%. Preferably, 2,2-dimethyl-1,3-dioxolane-4-methanol has an enantiomerica excess (ee)>80%, more preferably >90%, in particular >95%, more in particular >98%, most in particular >99%. Surprisingly, with the process of the invention, the ee of the starting product, the corresponding 2,2-dimethyl-1,3-dioxolane-4-methanol is almost equal to the ee of the glyceraldehyde acetonide prepared. This implies that hardly any racemisation occurs during the process of the invention.

In the scope of the invention, with an inert base is meant a base which does not react (apart from usual acid-base reactions) with the organic N-chloro compound or one of its degradation products, TEMPO or a TEMPO-derivative of formula 1, wherein R¹, R², R³, R⁴, R⁵ and R⁶ are as defined above, nor with glyceraldehyde acetonide or 2,2-dimethyl-1,3-dioxolane-4-methanol. The inert base is used in the process of the invention to neutralize the HCl that is formed in the reaction. Preferably a base, which has a conjugated acid with a pKₐ>2, is used. Examples of inert bases include sodium acetate and sodium bicarbonate.

Preferably an amount of base is used that is at least 0.8 molar equivalent based on the theoretically maximal molar amount of HCl that can be formed in the reaction, more preferably at least 0.9 molar equivalent, most preferably at least 1 molar equivalent.

The temperature of the process of the invention is in principle not critical. Preferably a temperature >−20° C., more preferably >0° C., even more preferably >15° C. is used. The temperature is preferably <100° C., more preferably <80° C. In practice, it is preferred to use a temperature between −20 and 100° C., more preferably between 15 and 80° C.

The order of addition of the organic N-chloro compound, TEMPO or a TEMPO-derivative of formula 1, wherein R¹-R⁶ are as defined above, is in principle not critical. Preferably, TEMPO or a TEMPO-derivative of formula 1, wherein R¹-R⁶ are as defined above, is added to a mixture of 2,2-dimethyl-1,3-dioxolane-4-methanol, the organic N-chloro compound and the inert base in a solvent.

Suitable organic N-chloro compounds include N-chloro-4-toluenesulphonamide sodium salt, N-chloro-benzensulphonamide sodium salt, trichloroisocyanuric acid and dichlorodimethylhydantoin. Preferably, the organic N-chloro compound is trichloroisocyanuric acid or dichlorodimethylhydantoin.

The amount of organic N-chloro compound is in principle not critical. It is preferred to use an amount of organic N-chloro compound such that there is at least 0.5 molar equivalent active chlorine based on the amount of 2,2-dimethyl-1,3-dioxolane-4-methanol, more preferably at least 1 molar equivalent active chlorine and most preferably at least 1.1 molar equivalent active chlorine. The maximal amount of organic N-chloro compound is in principle not critical. For economical reasons, the amount of active chlorine is preferably less than 5 molar equivalent based on the amount of 2,2-dimethyl-1,3-dioxolane-4-methanol.

The amount of TEMPO or a TEMPO-derivative of formula 1, wherein R¹-R⁶ are as defined above, used in the process of the invention, is in principle not critical. However, for economical reasons, preferably <20 mole %, more preferably <5 mole %, in particular <1 mole % TEMPO or a TEMPO-derivative (based on the amount of 2,2-dimethyl-1,3-dioxolane-4-methanol) is used. Preferably, the amount of TEMPO or a TEMPO-derivative (based on the amount of 2,2-dimethyl-1,3-dioxolane-4-methanol) is >0.01 mole %, more preferably >0.02 mole %, even more preferably >0.05 mole %, most preferably >0.1 mole %. In practice, it is preferred to use an amount of TEMPO or a TEMPO-derivative of between 0.1 and 1 mole % based on the amount of 2,2-dimethyl-1,3-dioxolane-4-methanol.

Preferably the process of the invention is carried out in the presence of a solvent.

Suitable solvents include ketones, for example acetone, 2-butanone, methyl isobutyl ketone; esters, for example ethyl acetate or methyl acetate; halogenated hydrocarbons, for example dichloromethane; ethers, for example methyl-t-butylether or diethylether; aromatic solvents, for example toluene; nitrites, for example acetonitrile; amides, for example N,N-dimethylformamide; lactams, for example N-methyl pyrrolidinone; sulfoxides, for example dimethylsulfoxide. The preference for a solvent depends amongst others on the solubility of the chosen base and can easily be determined by the person skilled in the art.

Glyceraldehyde acetonide, in particular (S)-glyceraldehyde acetonide, is a useful intermediate in the synthesis of, for instance drugs, in particular anti-viral drugs, agrochemicals and the like. WO03/022853 describes for instance a process for the preparation of the following compounds (in particular the preparation of several compounds in enantiomerically enriched form): (2-(2,2-dimethyl-[1,3]dioxolan-4-ylmethylene)-malonic acid diethyl ester; 2-[1-(2,2-dimethyl-[1,3]dioxolan-4-yl)-2-nitroethyl]-malonic acid dimethyl ester; 4-methoxy-2-oxo-hexahydro-furo[3,4-b]furan-3-carboxylic acid methyl ester; 2-(4-hydroxy-2-methoxy-tetrahydro-furan-3-yl)-malonic acid dimethyl ester; 4-methoxy-tetrahydro-furo[3,4-b]furan-2-one, 4-hydroxy-2-methoxy-tetrahydro-furan-3-yl) acetic acid methyl ester; hexahydro-furo[2,3-b]furan-3-ol) starting from (S)-glyceraldehyde acetonide. These compounds can be used, in particular in enantiomerically enriched form, in the preparation of anti-viral drugs, in particular anti-HIV drugs, more in particular HIV protease inhibitors. These compounds will be indicated below using the reference numbers as used in WO03/022853. The compounds are of particular interest in preparing HIV protease inhibitors as disclosed in WO 95/24385, WO99/65870, WO 00/47551, WO 00/76961 and U.S. Pat. No. 6,127,372, WO 01/25240, EP 0 715 618 and WO 99/67417 all incorporated herein by reference, and in particular in preparing the following HIV protease inhibitors:
[(1S,2R)-2-hydroxy-3-[[(4-methoxyphenyl)sulfonyl](2-methylpropyl)amino]-1-(phenylmethyl)propyl]-carbamic acid (3R,3aS,6aR)-hexahydrofuro[2,3-b]furan-3-yl ester (HIV protease inhibitor 1);
[(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester (HIV protease inhibitor 2);
[(1S,2R)-3-[(1,3-benzodioxol-5-ylsulfonyl)(2-methylpropyl)amino]-2-hydroxy-1-(phenylmethyl)propyl]-carbamic acid (3R,3aS,6aR)-hexahydrofuro[2,3-b]furan-3-yl ester (HIVprotease inhibitor 3), or any pharmaceutically acceptable addition salt thereof.

According to WO03/022853, 2-(2,2-dimethyl-[1,3]dioxolan-4-ylmethylene)-malonic acid diethyl ester (compound III.2) can be prepared from glyceraldehyde acetonide using dimethylmalonate. 2-[1-(2,2-dimethyl-[1,3]dioxolan-4-yl)-2-nitroethyl]-malonic acid dimethyl ester (compound III.3) can be prepared by reaction of 2-(2,2-dimethyl-[1,3]dioxolan-4-ylmethylene)-malonic acid diethyl ester (compound III.2) with nitromethane in the presence of a catalytic amount of 1,8-diazabicylclo[5.4.0]undec-7-ene (DBU). 4-Methoxy-2-oxo-hexahydro-furo[3,4-b]furan-3-carboxylic acid methyl ester (compound III.4) and 2-(4-hydroxy-2-methoxy-tetrahydro-furan-3-yl)-malonic acid dimethyl ester (compound III.4′) can be prepared from 2-[1-(2,2-dimethyl-[1,3]dioxolan-4-yl)-2-nitroethyl]-malonic acid dimethyl ester (compound III.3) by using first a base and subsequently an acid. The compounds 4-methoxy-tetrahydro-furo[3,4-b]furan-2-one (compound III.5) and 4-hydroxy-2-methoxy-tetrahydro-furan-3-yl) acetic acid methyl ester (compound III.5′) may be prepared by decarboxylation of the compounds 4-methoxy-2-oxo-hexahydro-furo[3,4-b]furan-3-carboxylic acid methyl ester (compound III.4) and 2-(4-hydroxy-2-methoxy-tetrahydro-furan-3-yl)-malonic acid dimethyl ester (compound III.4′). Hexahydro-furo[2,3-b]furan-3-ol (compound 7.1) can be prepared by reduction of 4-methoxy-tetrahydro-furo[3,4-b]furan-2-one (compound III.5), which results in the intermediate compound: 4-(2-hydroxy-ethyl)-5-methoxy-tetrahydro-furan-3-ol (compound of formula (6)), which can then be cyclisized to form hexahydro-furo[2,3-b]furan-3-ol (compound 7.1.).

The process of the invention will now be elucidated by way of the following examples without however being limited thereto.

### EXAMPLE 1

### Screening of Oxidants

Materials and Methods

(R,S)-Solketal (=(R,S)-2,2-dimethyl-1,3-dioxolane-4-methanol, 97 wt %) is used as received from Acros. 4-Methylmorpholine-N-oxide (monohydrate), pyridine-N-oxide (as applied in Expreiments E-H), RuCl₃, ZrOCl₂, TEMPO (=2,2,6,6-tetramethylpiperidinyloxy radical), TCCA (=trichloroisocyanuric acid, 1 mole corresponds with 3 moles active chlorine) and NaOCl (aqueous solution, containing 12 wt % active chlorine) are used as received from Aldrich. Acetonitrile of p.a. quality (Merck) is used without further purification. H₂O₂ (50 wt % aqueous solution) is used as received from Degussa. Pyridine-N-oxide (as used in experiments I-L) is prepared in situ by treating a solution of pyridine (p.a. quality, Merck) in CHCl₃ (p.a. quality, Merck) at 0° C. with H₂O₂ (10 molar equivalent based on pyridine, 50 wt % aqueous solution, Degussa), stirring the reaction mixture overnight, separating the organic phase, washing it once with water and evaporating the solvent to give a residue which is used in the oxidation reaction without further purification.

For the GC analysis we used an Agilent 6890 GC combined with a DB-5 column with a length of 10 m, an internal diameter of 0.10 mm and a film thickness of 0.10 μm and using a split-flow inlet system (helium as carrier gas, column flow 0.5 mL/min and split 100:1). The injection volume was 1.0 μL. For detection an FID was used at 300° C. The injection temperature was 250° C. and the column temperature programme consisted of 0.5 min at 100° C. followed by an increase to 280° C. with 40° C./min.

General Procedure

0.50 g (3.79 mmole) (R,S)-Solketal is dissolved in 5 mL acetonitrile and subsequently 0.38 mmole (=0.1 molar equivalent based on (R,S)-Solketal) of the catalyst (as shown in Table A) is added. To this solution was added (at room temperature and during 30 min.) a solution of the oxidant (1 or 2 molar equivalent based on (R,S)-Solketal, as shown in Table A). In case of 4-methylmorpholine-N-oxide, pyridine-N-oxide and TCCA the oxidant is added as a solution in 2 mL acetonitrile, in case of Pyr-NO, NaOCl and H₂O₂ the oxidant is added as an aqueous solution. The reaction mixture is stirred at room temperature and the (R,S)-Solketal conversion to (R,S)-Solketalaldehyde is monitored by GC. The maximal conversion measured is displayed in Table A.

Results

TABLE A
The conversion is defined as area % (R,S)-Solketalaldehyde
divided by [area % (R,S)-Solketal + area %
(R,S)-Solketalaldehyde] from the GC chromatogram.

| | | Molar | | Conver- |
|---|---|---|---|---|
| Experi- | | equivalent | | sion |
| ment | Oxidant | oxidant* | Catalyst | (%) |
| A | 4-methylmorpholine-N-oxide | 1 | RuCl₃ | 7 |
| B | 4-methylmorpholine-N-oxide | 2 | RuCl₃ | 11 |
| C | 4-methylmorpholine-N-oxide | 1 | TEMPO | 2 |
| D | 4-methylmorpholine-N-oxide | 2 | TEMPO | 3 |
| E | Pyridine-N-oxide | 1 | ZrOCl₂ | 0 |
| F | Pyridine-N-oxide | 2 | ZrOCl₂ | <1 |
| G | Pyridine-N-oxide | 1 | TEMPO | 0 |
| H | Pyridine-N-oxide | 2 | TEMPO | 0 |
| I | Pyridine-N-oxide** | 1 | RuCl₃ | <1 |
| J | Pyridine-N-oxide** | 2 | RuCl₃ | <1 |
| K | Pyridine-N-oxide** | 1 | ZrOCl₂ | 0 |
| L | Pyridine-N-oxide** | 2 | ZrOCl₂ | 0 |
| M | NaOCl | 1 | RuCl₃ | 0 |
| N | NaOCl | 2 | RuCl₃ | <1 |
| O | NaOCl | 1 | ZrOCl₂ | 12 |
| P | NaOCl | 2 | ZrOCl₂ | 11 |
| Q | NaOCl | 1 | TEMPO | <1 |
| R | NaOCl | 2 | TEMPO | <1 |
| S | H₂O₂ | 1 | ZrOCl₂ | <1 |
| T | H₂O₂ | 2 | ZrOCl₂ | 12 |
| U | TCCA | 0.4 | TEMPO | 44 |

*based on (R,S)-Solketal
**prepared in situ (see materials and methods)

In conclusion, the above screening of oxidants shows that the combination of TCCA (an organic N-chloro compound) with TEMPO (or a TEMPO-derivative) used in the process of the invention (example U) is the only combination of oxidizing agent with catalyst that leads to an acceptable conversion of 2,2-dimethyl-1,3-dioxolane-4-methanol to glyceraldehyde acetonide.

### EXAMPLE 2

### Influence of Parameters

Materials and methods

(R)-Solketal (=(R)-2,2-dimethyl-1,3-dioxolane-4-methanol, 97 wt %, e.e. 99.6%), (R,S)-Solketal (=(R,S)-2,2-dimethyl-1,3-dioxolane-4-methanol, 97 wt %), TCCA (=trichloroisocyanuric acid, 99 wt %, 1 mole corresponds with 3 moles active chlorine), DCDMH (=dichlorodimethylhydantoin, 1 mole corresponds with 1.33 moles active chlorine), TEMPO (=2,2,6,6-tetramethylpiperidinyloxy radical, 98 wt %), NaOAc (=sodium acetate, 99 wt %), NaHCO₃ (=sodium bicarbonate, 99 wt %), K₂CO₃ (=potassium carbonate, 99 wt %) and TEPA (=triethyl phosphonoacetate, 97 wt %) are used as received from Acros, as well as the solvents acetone, 2-butanone, ethyl acetate, acetonitrile and dichloromethane (all of p.a. quality).

For the GC analysis we used an Agilent 6890 GC (EPC) and an Agilent 7683 Automatic Liquid Sampler combined with a Betadex column (part number 24305, Supelco) with a length of 60 m, an internal diameter of 0.25 mm and a film thickness of 0.25 μm and using a split-flow inlet system with constant flow (Helium as carrier gas, column head pressure 26.4 kPa, column flow 1.4 mL/min and split flow 37.5 mL/min). For detection an FID was used at 250° C. For the quantitative analytical determination of (R)- and (S)-glyceraldehyde acetonide the injection temperature was 150° C. and the column temperature programme consisted of 3 min at 60° C., increase to 130° C. with 5° C./min, another 1 min at 130° C. and increase to 230° C. with 25° C./min. For the quantitative analytical determination of (R)- and (S)-ethyl-((4,5)-O-isopropylidene-(4,5)-dihydroxy)-2-(E,Z)-pentenoate the injection temperature was 250° C. and the column temperature programme consisted of 1 min at 80° C., increase to 225° C. with 5° C./min and another 10 min at 225° C.

Influence of the Solvent

TABLE 1
Influence of the solvent using 120 mole % NaOAc,
40 mole % TCCA and 0.25 mole % TEMPO based on Solketal.

| | | Yield (%) of glyceraldehyde acetonide |
|---|---|---|
| Example | Solvent | (based on Solketal) |
| 2.1 | Acetone | 80 |
| 2.2 | 2-Butanone | 77 |
| 2.3 | Ethyl acetate | 67 |
| 2.4 | Acetonitrile | 64 |
| 2.5 | Dichloromethane | 61 |

### EXAMPLE 2.1

### Oxidation of (R)-Solketal to (S)-glyceraldehyde acetonide in acetone

In a 100 ml 4-necked round-bottomed flask, 5.46 g (40.0 mmole) (R)-Solketal is dissolved in 25.9 g acetone. 3.95 g (48.2 mmole) NaOAc and 3.72 g (16.0 mmole) TCCA are subsequently added and the resulting suspension is stirred at 25° C.

A solution of 15.7 mg (0.10 mmole) TEMPO in 11.3 g acetone is added over 8 minutes, allowing the reaction mixture to warm up to 59° C. The reaction mixture is stirred during another 30 minutes and the solids are filtered off and washed with 25 g acetone. GC analysis of the filtrate demonstrates that 4.2 g (32.2 mmole, 80% yield based on (R)-Solketal) (S)-glyceraldehyde acetonide (e.e.=99.5%) has been obtained.

### EXAMPLE 2.2

### Oxidation of (R,S)-Solketal to (R,S)-glyceraldehyde acetonide in 2-butanone

In a 100 ml 4-necked round-bottomed flask, 4.09 g (30.0 mmole) (R,S)-Solketal is dissolved in 18.9 g 2-butanone. 2.96 g (36.1 mmole) NaOAc and 2.79 g (12.0 mmole) TCCA are subsequently added and the resulting suspension is stirred at 25° C. A solution of 11.8 mg (0.075 mmole) TEMPO in 8.5 g 2-butanone is added over 9 minutes, allowing the reaction mixture to warm up to 74° C. The reaction mixture is stirred during another 30 minutes and the solids are filtered off and washed with 20 g 2-butanone. GC analysis of the filtrate demonstrates that 3.0 g (23.2 mmole, 77% yield based on (R,S)-Solketal) (R,S)-glyceraldehyde acetonide has been obtained.

### EXAMPLE 2.3

### Oxidation of (R,S)-Solketal to (R,S)-glyceraldehyde acetonide in ethyl acetate

A solution of 4.09 g (30.0 mmole) (R,S)-Solketal in 19.0 g ethyl acetate is oxidized in ethyl acetate according to the procedure of example 2.2 except that the TEMPO solution is added over 10 minutes, allowing the reaction mixture to warm up to 77° C. and that the solids are washed with ethyl acetate. GC analysis of the filtrate demonstrates that 2.6 g (20.2 mmole, 67% yield based on (R,S)-Solketal) (R,S)-glyceraldehyde acetonide has been obtained.

### EXAMPLE 2.4

### Oxidation of (R,S)-Solketal to (R,S)-glyceraldehyde acetonide in acetonitrile

A solution of 4.09 g (30.0 mmole) (R,S)-Solketal in 19.2 g acetonitrile is oxidized in acetonitrile according to the procedure of example 2.2 except that the TEMPO solution is added over 6 minutes, allowing the reaction mixture to warm up to 60° C. and that the solids are washed with acetonitrile. GC analysis of the filtrate demonstrates that 2.5 g (19.1 mmole, 64% yield based on (R,S)-Solketal) (R,S)-glyceraldehyde acetonide has been obtained.

### EXAMPLE 2.5

### Oxidation of (R,S)-Solketal to (R,S)-glyceraldehyde acetonide in dichloromethane

A solution of 4.09 g (30.0 mmole) (R,S)-Solketal in 28.0 g dichloromethane is oxidized in dichloromethane according to the procedure of example 2.2 except that the TEMPO solution is added over 15 minutes, allowing the reaction mixture to warm up to 42° C. and that the solids are washed with 30 g dichloromethane. GC analysis of the filtrate demonstrates that 2.4 g (18.4 mmole, 61% yield based on (R,S)-Solketal) (R,S)-glyceraldehyde acetonide has been obtained.

Influence of the Amount of TEMPO

TABLE 2
Influence of the amount of TEMPO using 120
mole % NaOAc and 40 mole % TCCA based on
Solketal and using acetone as the solvent.

| | TEMPO | Yield (%) of glyceraldehyde |
|---|---|---|
| Example | (mole % based on Solketal) | acetonide (based on Solketal) |
| 2.6 | 1.0 | 74 |
| 2.7 | 0.33 | 77 |
| 2.1 | 0.25 | 80 |
| 2.8 | 0.15 | 74 |
| 2.9 | 0.081 | 70 |
| 2.10 | 0.023 | 60 |

### EXAMPLE 2.6

### Oxidation of (R,S)-Solketal to (R,S)-glyceraldehyde acetonide with 1.0 Mole % TEMPO

In a 100 ml 4-necked round-bottomed flask, 4.09 g (30.0 mmole) (R,S)-Solketal is dissolved in 18.0 g acetone. 2.96 g (36.1 mmole) NaOAc and 2.79 g (12.0 mmole) TCCA are subsequently added and the resulting suspension is stirred at 25° C. A solution of 48.0 mg (0.30 mmole) TEMPO in 12.2 g acetone is added over 7 minutes, allowing the reaction mixture to warm up to 59° C. The reaction mixture is stirred during another 30 minutes and the solids are filtered off and washed with 20 g acetone. GC analysis of the filtrate demonstrates that 2.9 g (22.1 mmole, 74% yield based on (R,S)-Solketal) (R,S)-glyceraldehyde acetonide has been obtained.

### EXAMPLE 2.7

### Oxidation of (R,S)-Solketal to (R,S)-glyceraldehyde acetonide with 0.33 Mole % TEMPO

A solution of 4.10 g (30.1 mmole) (R,S)-Solketal in 19.3 g acetone is oxidized according to the procedure of example 2.6, except that a solution of 15.8 mg (0.10 mmole) TEMPO in 9.3 g acetone is added over 12 minutes, allowing the reaction mixture to warm up to 60° C. GC analysis of the filtrate demonstrates that 3.0 g (23.1 mmole, 77% yield based on (R,S)-Solketal) (R,S)-glyceraldehyde acetonide has been obtained.

### EXAMPLE 2.8

### Oxidation of (R,S)-Solketal to (R,S)-glyceraldehyde acetonide with 0.15 Mole % TEMPO

A solution of 5.45 g (40.0 mmole) (R,S)-Solketal in 25.6 g acetone is oxidized according to the procedure of example 2.1, except that a solution of 9.4 mg (0.059 mmole) TEMPO in 9.3 g acetone is added over 11 minutes, allowing the reaction mixture to warm up to 59° C. GC analysis of the filtrate demonstrates that 3.8 g (29.6 mmole, 74% yield based on (R,S)-Solketal) (R,S)-glyceraldehyde acetonide has been obtained.

### EXAMPLE 2.9

### Oxidation of (R,S)-Solketal to (R,S)-glyceraldehyde acetonide with 0.081 Mole % TEMPO

In a 100 ml 4-necked round-bottomed flask, 6.14 g (45.0 mmole) (R,S)-Solketal is dissolved in 30.2 g acetone. 4.44 g (54.23 mmole) NaOAc and 4.19 g (18.0 mmole) TCCA are subsequently added and the resulting suspension is stirred at 25° C. A solution of 5.8 mg (0.036 mmole) TEMPO in 12.3 g acetone is added over 10 minutes, allowing the reaction mixture to warm up to 59° C. The reaction mixture is stirred during another 30 minutes and the solids are filtered off and washed with 28 g acetone. GC analysis of the filtrate demonstrates that 4.1 g (31.5 mmole, 70% yield based on (R,S)-Solketal) (R,S)-glyceraldehyde acetonide has been obtained.

### EXAMPLE 2.10

### Oxidation of (R,S)-Solketal to (R,S)-glyceraldehyde acetonide with 0.023 Mole % TEMPO

A solution of 4.10 g (30.1 mmole) (R,S)-Solketal in 19.7 g acetone is oxidized according to the procedure of example 2.6, except that a solution of 1.1 mg (0.0068 mmole) TEMPO in 8.6 g acetone is added over 10 minutes, allowing the reaction mixture to warm up to 46° C. GC analysis of the filtrate demonstrates that 2.3 g (18.0 mmole, 60% yield based on (R,S)-Solketal) (R,S)-glyceraldehyde acetonide has been obtained.

Influence of the Amount of TCCA

TABLE 3
Influence of the amount of TCCA using 0.25
mole % TEMPO based on Solketal and 300 mole % NaOAc
based on TCCA and using acetone as the solvent.

| | | Yield (%) of glyceraldehyde |
|---|---|---|
| | TCCA | acetonide (based on |
| Example | (mole % based on Solketal) | Solketal) |
| 2.11 | 33 | 73 |
| 2.1 | 40 | 80 |
| 2.12 | 50 | 77 |
| 2.13 | 100 | 67 |

### EXAMPLE 2.11

### Oxidation of (R,S)-Solketal to (R,S)-glyceraldehyde acetonide with 33 Mole % TCCA

In a 100 ml 4-necked round-bottomed flask, 4.10 g (30.1 mmole) (R,S)-Solketal is dissolved in 19.2 g acetone. 2.51 g (30.3 mmole) NaOAc and 2.35 g (10.0 mmole) TCCA (containing 30.0 mmole active chlorine) are subsequently added and the resulting suspension is stirred at 25° C. A solution of 12.3 mg (0.077 mmole) TEMPO in 8.6 g acetone is added over 8 minutes, allowing the reaction mixture to warm up to 59° C. After the TEMPO addition is complete, the reaction mixture is cooled to 25° C over 5 minutes. The solids are filtered off and washed with 25 g acetone. GC analysis of the filtrate demonstrates that 2.9 g (21.9 mmole, 73% yield based on (R,S)-Solketal) (R,S)-glyceraldehyde acetonide has been obtained.

### EXAMPLE 2.12

### Oxidation of (R,S)-Solketal to (R,S)-glyceraldehyde acetonide with 50 Mole % TCCA

A solution of 4.09 g (30.0 mmole) (R,S)-Solketal in 22.2 g acetone is oxidized according to the procedure of example 2.11, except that 3.77 g (45.45 mmole) NaOAc and 3.52 g (15.0 mmole) TCCA (containing 45.0 mmole active chlorine) are used and that the TEMPO solution is added over 12 minutes, allowing the reaction mixture to warm up to 59° C. GC analysis of the filtrate demonstrates that 3.0 g (23.0 mmole, 77% yield based on (R,S)-Solketal) (R,S)-glyceraldehyde acetonide has been obtained.

### EXAMPLE 2.13

### Oxidation of (R,S)-Solketal to (R,S)-glyceraldehyde acetonide with 100 mole % TCCA

In a 100 mL 4-necked round-bottomed flask, 4.44 g (32.6 mmole) (R,S)-Solketal is dissolved in 25.3 g acetone. 8.16 g (98.5 mmole) NaOAc and 7.65 g (32.6 mmole) TCCA (containing 97.8 mmole active chlorine) are subsequently added and the resulting suspension is stirred at 25° C. A solution of 13.0 mg (0.082 mmole) TEMPO in 9.7 g acetone is added over 9 minutes, allowing the reaction mixture to warm up to 59° C. GC analysis of the filtrate demonstrates that 2.9 g (21.9 mmole, 67% yield based on (R,S)-Solketal) (R,S)-glyceraldehyde acetonide has been obtained.

Influence of the Amount of NaOAc Relative to TCCA

TABLE 4
Influence of the amount of NaOAc based on
TCCA using 40 mole % TCCA and 0.25 mole % TEMPO
based on Solketal and using acetone as the solvent.

| | NaOAc | Yield (%) of glyceraldehyde |
|---|---|---|
| Example | (mole % based on Solketal) | acetonide (based on Solketal) |
| 2.14 | 80 | 37 |
| 2.15 | 108 | 73 |
| 2.1 | 120 | 80 |

### EXAMPLE 2.14

### Oxidation of (R,S)-Solketal to (R,S)-glyceraldehyde acetonide with 80 Mole % NaOAc

In a 100 ml 4-necked round-bottomed flask, 4.09 g (30.0 mmole) (R,S)-Solketal is dissolved in 19.3 g acetone. 2.00 g (24.1 mmole) NaOAc and 2.83 g (12.0 mmole) TCCA are subsequently added and the resulting suspension is stirred at 25° C. A solution of 12.0 mg (0.075 mmole) TEMPO in 8.9 g acetone is added over 9 minutes, allowing the reaction mixture to warm up to 57° C. After the TEMPO addition is complete, the reaction mixture is stirred during another 30 minutes. The solids are filtered off and washed with 25 g acetone. GC analysis of the filtrate demonstrates that 1.4 g (11.1 mmole, 37% yield based on (R,S)-Solketal) (R,S)-lyceraldehyde acetonide has been obtained.

### EXAMPLE 2.15

### Oxidation of (R,S)-Solketal to (R,S)-glyceraldehyde acetonide with 108 Mole % NaOAc

A solution of 4.09 g (30.0 mmole) (R,S)-Solketal in 19.5 g acetone is oxidized according to the procedure of example 2.14, except that 2.66 g (32.1 mmole) NaOAc is used. After the addition of the TEMPO solution the temperature of the reaction mixture rose to 59° C. GC analysis of the filtrate demonstrates that 2.8 g (21.7 mmole, 73% yield based on (R,S)-Solketal) (R,S)-glyceraldehyde acetonide has been obtained.

Influence of the Type of Base

TABLE 5
Influence of the type of base using 40 mole %
TCCA and 120 mole % base based on Solketal

| | | | Yield (%) of glyceraldehyde |
|---|---|---|---|
| | | | acetonide |
| Example | Base | Solvent | (based on Solketal) |
| 2.1 | NaOAc | Acetone | 80 |
| 2.5 | NaOAc | Dichloromethane | 61 |
| 2.16 | NaHCO₃ | Dichloromethane | 44 |

### EXAMPLE 2.16

### Oxidation of (R)-Solketal to (S)-glyceraldehyde acetonide with NaHCO₃ in dichloromethane

In a 100 ml 4-necked round-bottomed flask, 4.19 g (30.7 mmole) (R)-Solketal is dissolved in 24.8 g dichloromethane. 3.07 g (36.2 mmole) NaHCO₃ and 2.84 g (12.1 mmole) TCCA are subsequently added and the resulting suspension is cooled to 3° C. A solution of 24.1 mg (0.151 mmole) TEMPO in 10.6 g dichloromethane is added over 15 minutes at 3-8° C. The reaction mixture is stirred during 60 minutes at 5° C. and the solids are filtered off and washed with 25 g dichloromethane. GC analysis of the filtrate demonstrates that 1.8 g (13.6 mmole, 44% yield based on (R)-Solketal) (S)-glyceraldehyde acetonide (e.e. =99.6%) has been obtained.

Influence of the Addition Time of the TEMPO Solution

TABLE 6
Influence of the addition time of the TEMPO solution
using 120 mole % NaOAc, 40 mole % TCCA and
0.25 mole % TEMPO based on Solketal and using
acetone as the solvent

| | Dosing time | Yield (%) of glyceraldehyde acetonide |
|---|---|---|
| Example | (min.) | (based on Solketal) |
| 2.17 | 1.4 | 78 |
| 2.1 | 8 | 80 |

### EXAMPLE 2.17

### Oxidation of (R,S)-Solketal to (R,S)-glyceraldehyde acetonide with Fast Addition of TEMPO

In a 100 ml 4-necked round-bottomed flask, 4.09 g (30.0 mmole) (R,S)-Solketal is dissolved in 20.2 g acetone. 2.99 g (36.1 mmole) NaOAc and 2.82 g (12.0 mmole) TCCA are subsequently added and the resulting suspension is stirred at 25° C. A solution of 12.0 mg (0.075 mmole) TEMPO in 6.4 g acetone is added over 85 seconds, allowing the reaction mixture to warm up to 59° C. The reaction mixture is cooled to 22° C and the solids are filtered off and washed with 25 g acetone. GC analysis of the filtrate demonstrates that 3.0 g (23.4 mmole, 78% yield based on (R,S)-Solketal) (R,S)-glyceraldehyde acetonide has been obtained.

Influence of Temperature

TABLE 7
Influence of the reaction temperature using 120
mole % NaOAc, 40 mole % TCCA and 0.25 mole % TEMPO
based on Solketal and using acetone as the solvent

| | | Yield (%) of glyceraldehyde acetonide |
|---|---|---|
| Example | Temperature | (based on Solketal) |
| 2.18 | 6-15° C. | 59 |
| 2.1 | 25-59° C. | 80 |

### EXAMPLE 2.18

### Oxidation of (R)-Solketal to (S)-glyceraldehyde acetonide at Low Temperature

In a 100 ml 4-necked round-bottomed flask, 4.10 g (30.1 mmole) (R)-Solketal is dissolved in 24.4 g acetone. 2.99 g (36.1 mmole) NaOAc is added and the suspension is cooled to 1° C. 2.83 g (12.0 mmole) TCCA is added, while the temperature is kept below 4° C. The resulting suspension is further cooled to 0° C. and subsequently, a solution of 12.1 mg (0.076 mmole) TEMPO in 7.4 g acetone is added over 9 minutes. The reaction mixture is allowed to reach a maximum temperature of 24° C., but during at least 90% of the addition time of the TEMPO solution the temperature was between 15 and 6° C. After complete addition of the TEMPO solution, the reaction mixture is stirred during 60 minutes between 0 and 6° C. and subsequently warmed up to 20° C. during 60 minutes. The solids are filtered off and washed with 25 g acetone. GC analysis of the filtrate demonstrates that 2.3 g (17.8 mmole, 59% yield based on (R)-Solketal) (S)-glyceraldehyde acetonide (e.e.=99.6%) has been obtained.

Influence of Addition Order

TABLE 8
Influence of the addition order using 120 mole
% NaOAc, 40 mole % TCCA and 0.25 mole % TEMPO
based on Solketal and using acetone as the solvent

| | | Yield (%) glyceraldehyde acetonide |
|---|---|---|
| Example | Dosed reagent | (based on Solketal) |
| 2.19 | TCCA | 67 |
| 2.1 | TEMPO | 80 |

### EXAMPLE 2.19

### Oxidation of (R,S)-Solketal to (R,S)-glyceraldehyde acetonide Using Addition of TCCA Instead of TEMPO

In a 100 ml 4-necked round-bottomed flask, 4.21 g (30.9 mmole) (R,S)-Solketal is dissolved in 16.6 g acetone. 3.09 g (37.3 mmole) NaOAc and 12.3 mg (0.077 mmole) TEMPO are subsequently added and the resulting suspension is stirred at 25° C. A solution of 2.90 g (12.4 mmole) TCCA acid in 22.6 g acetone is added over 15 minutes, allowing the reaction mixture to warm up to 47° C. The reaction mixture is stirred during another 60 minutes and the solids are filtered off and washed with 25 g acetone. GC analysis of the filtrate demonstrates that 2.7 g (20.7 mmole, 67% yield based on (R,S)-Solketal) (R,S)-glyceraldehyde acetonide has been obtained.

Influence of the Type of Oxidant

TABLE 9
Influence of the type of oxidant using 0.25 mole %
TEMPO based on Solketal and using acetone as the
solvent and NaOAc as the base

| | | | | Yield (%) of |
|---|---|---|---|---|
| | Oxidizing | Amount | Active chlorine | glyceraldehyde |
| Example | agent | (mole %*) | (mole %*) | acetonide* |
| 2.11 | TCCA | 33 | 100 | 73 |
| 2.1 | TCCA | 40 | 120 | 80 |
| 2.12 | TCCA | 50 | 150 | 77 |
| 2.13 | TCCA | 100 | 300 | 67 |
| 2.20 | DCDMH | 90 | 120 | 72 |
| 2.21 | DCDMH | 120 | 160 | 65 |

*Based on Solketal

### EXAMPLE 2.20

### Oxidation of (R)-Solketal to (S)-glyceraldehyde acetonide with 90 Mole % DCDMH

In a 100 ml 4-necked round-bottomed flask, 4.11 g (30.1 mmole) (R)-Solketal is dissolved in 30.1 g acetone. 6.73 g (81.2 mmole) NaOAc and 5.33 g (27.0 mmole) DCDMH (containing 36.0 mmole active chlorine) are subsequently added and the resulting suspension is stirred at 18° C. A solution of 12.2 mg (0.077 mmole) TEMPO in 8.7 g acetone is added over 9 minutes, allowing the reaction mixture to warm up to 51° C. The reaction mixture is stirred during another 30 minutes and the solids are filtered off and washed with 25 g acetone. GC analysis of the filtrate demonstrates that 2.8 g (21.6 mmole, 72% yield based on (R)-Solketal) (S)-glyceraldehyde acetonide (e.e.=99.6%) has been obtained.

### EXAMPLE 2.21

### Oxidation of (R)-Solketal to (S)-glyceraldehyde acetonide with 120 Mole % DCDMH

A solution of 4.10 g (30.0 mmole) (R)-Solketal in 30.5 g acetone was oxidized according to the procedure of example 2.20, except that 5.98 g (72.2 mmole) NaOAc and 7.10 g (36.0 mmole) DCDMH (containing 48.0 mmole active chlorine) were used and that the TEMPO solution is added over 10 minutes, allowing the reaction mixture to warm up to 57° C. GC analysis of the filtrate demonstrates that 2.5 g (19.4 mmole, 65% yield based on (R)-Solketal) (S)-glyceraldehyde acetonide (e.e.=99.5%) has been obtained.

Influence of the Reaction Conditions on the e.e.

TABLE 10
Influence of the reaction conditions on the e.e.

| | | | Active | | | Yield (%) of | |
|---|---|---|---|---|---|---|---|
| | | | chlorine | TEMPO | | glyceraldehyde | E.e. |
| Ex. | Solvent | Oxidizing agent | (mole %*) | (mole %*) | T (° C.) | acetonide* | (%) |
| 2.1 | Acetone | TCCA | 120 | 0.25 | 25-59 | 80 | 99.5 |
| 2.16 | Dichloro- | TCCA | 120 | 0.50 | 3-8 | 44 | 99.6 |
| | methane | | | | | | |
| 2.18 | Acetone | TCCA | 120 | 0.25 | 0-24 | 59 | 99.6 |
| 2.20 | Acetone | DCDMH | 120 | 0.25 | 18-51 | 72 | 99.6 |
| 2.21 | Acetone | DCDMH | 160 | 0.25 | 18-57 | 65 | 99.5 |
| 2.22 | Acetone | TCCA | 180 | 0.50 | 25-58 | 71 | 99.6 |

*Based on Solketal

As can be seen from Table 10, starting from enantiomerically enriched solketal with an enantiomeric excess (e.e.) of 99.6, enantiomerically enriched glyceraldehyde acetonide with similar e.e. can be prepared.

### EXAMPLE 2.22

### Oxidation of (R)-Solketal to (S)-glyceraldehyde acetonide with More TEMPO and TCCA

In a 100 ml 4-necked round-bottomed flask, 4.14 g (30.3 mmole) (R)-Solketal is dissolved in 19.1 g acetone. 4.49 g (54.2 mmole) NaOAc and 4.25 g (18.1 mmole) TCCA are subsequently added and the resulting suspension is stirred at 25° C. A solution of 24.3 mg (0.15 mmole) TEMPO in 9.0 g acetone is added over 12 minutes, allowing the reaction mixture to warm up to 58° C. The reaction mixture is stirred during another 105 minutes and the solids are filtered off and washed with 25 g acetone. GC analysis of the filtrate demonstrates that 2.8 g (21.5 mmole, 71% yield based on (R)-Solketal) (S)-glyceraldehyde acetonide (e.e.=99.6%) has been obtained.

### EXAMPLE 2.23

### Preparation of (R)-ethyl-((4,5)-O-isopropylidene-(4,5)-dihydroxy)-2-(E,Z)-pentenoate

In a 250 ml 4-necked round-bottomed flask, 32.0 g (231.5 mmole) K₂CO₃ is dissolved in 120 ml water. The pH of this solution is adjusted to 11.5 by adding aq. 4 M HCl. The resulting solution is cooled to 3° C. and 8.0 g (34.6 mmole) TEPA is added. While maintaining the temperature of the reaction mixture below 5° C., 73.5 g of a solution containing 4.2 g (32.0 mmole) (S)-glyceraldehyde acetonide in acetone (as obtained by the procedure in example 2.1) is added over 70 minutes. The pH of the reaction mixture is meanwhile kept in the range 11.0-11.5 by adding small portions of K₂CO₃. The resulting mixture is stirred during 4 h between 0 and 5° C. and subsequently during 16 h between 5 and 15° C. GC analysis of the organic phase demonstrates that 5.8 g (28.8 mmole, 90% yield based on (S)-glyceraldehyde acetonide) (R)-ethyl-((4,5)-O-isopropylidene-(4,5)-dihydroxy)-2-(E,Z)-pentenoate (e.e.=97.8%, E/Z=95/5) has been obtained.

In conclusion, as is shown by example 2, with the process of the invention glyceraldehyde acetonide can be prepared with a high yield (higher than 30%) and with a high e.e.

1. Process for the preparation of glyceraldehyde acetonide by oxidation of 2,2-dimethyl-1,3-dioxolane-4-methanol by an oxidizing agent, characterized in that 2,2-dimethyl-1,3-dioxolane-4-methanol is oxidized by an organic N-chloro compound in the presence of an inert base and TEMPO or a TEMPO-derivative of formula 1 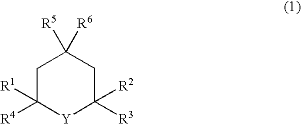
wherein R¹, R², R³ and R⁴ each independently stand for an alkyl group with 1 to 6 C-atoms and wherein R⁵ and R⁶ either both stand for H or an alkoxy group with 1 to 6 C-atoms or one stands for H and the other stands for an alkoxy group with 1 to 6 C-atoms, an alkylcarbonyloxy group with 1 to 6 C-atoms, an arylcarbonyloxy group with the carbonyloxy group having 1 to 6 C-atoms or an alkylcarbonylamino group with 1 to 6 C-atoms; or wherein R⁵ and R⁶ together stand for ketal groups of formula a-c 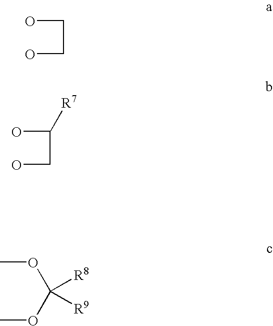
wherein R⁷ stands for an alkyl group with 1 to 6 C-atoms and R⁸ and R⁹ each independently stand for H or an alkyl group with 1 to 6 C-atoms and wherein Y stands for a group of general formula d-f 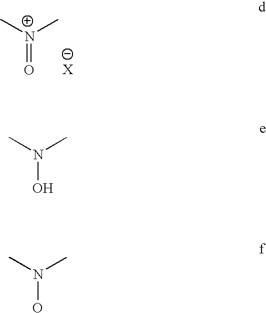
wherein X⁻ for an anion.

2. Process according to claim 1, characterized in that enantiomerically enriched glyceraldehyde acetonide is prepared by oxidation of the corresponding enantiomerically enriched 2,2-dimethyl-1,3-dioxolane-4-methanol.

3. Process according to claim 1, characterized in that the organic N-chloro compound is trichloroisocyanuric acid or dichlorodimethylhydantoin.

4. Process according to claim 1, characterized in that 2,2-dimethyl-1,3-dioxolane-4-methanol is oxidized in the presence of TEMPO.

5. Process according to claim 1, characterized in that the inert base has a conjugated acid with a pKₐ>2.

6. Process according to claim 1, characterized in that the amount of inert base is at least 0.8 molar equivalent based on the theoretically maximal molar amount of HCl that can be formed in the reaction.

7. Process according to claim 1, characterized in that the inert base is sodium acetate or sodium bicarbonate.

8. Process according to claim 1, characterized in that the process is performed at a temperature between 15 and 80° C.

9. Process according to claim 1, characterized in that the TEMPO or a TEMPO-derivative of formula 1, wherein R¹—R⁶ are as defined above, is added to a mixture of 2,2-dimethyl-1,3-dioxolane-4-methanol, the organic N-chloro compound and the inert base in a solvent.

10. Process according to claim 1, characterized in that the amount of organic N-chloro compound is such that there is at least 0.5 molar equivalent active chlorine based on the amount of 2,2-dimethyl-1,3-dioxolane-4-methanol.

11. Process according to claim 1, characterized in that an amount of TEMPO or a TEMPO-derivative of formula 1, wherein R¹-R⁶ are as defined above, of between 0.1 and 1 mole % based on the amount of 2,2-dimethyl-1,3-dioxolane-4-methanol is used.
